(19) United States
(12) Patent Application Publication
(10) Pub. No.: US 2014/0316243 A1
(43) Pub. Date: Oct. 23, 2014

(54) SYSTEMS AND TECHNIQUES FOR DELIVERY AND MEDICAL SUPPORT

(71) Applicant: Brian J. Niedermeyer, Bozeman, MT (US)

(72) Inventor: Brian J. Niedermeyer, Bozeman, MT (US)

(21) Appl. No.: 14/255,876

(22) Filed: Apr. 17, 2014

Related U.S. Application Data
(60) Provisional application No. 61/813,068, filed on Apr. 17, 2013.

(51) Int. Cl. A61B 5/00; A61B 19/00; G05D 1/00

(52) U.S. Cl. CPC A61B 5/7264; G05D 1/0088; A61B 5/0082; A61B 5/742; A61B 19/5244
USPC 600/408; 701/26; 600/407; 600/424

(57) ABSTRACT

Systems and techniques for delivery and medical support are disclosed herein. In some embodiments, a drone delivery system may include receiving logic and communication logic. The receiving logic may be configured to receive a request signal indicative of a package request event proximate to a target device, wherein the request signal comprises sensor data indicative of conditions proximate to the target device or a request signal transmitted to the receiving device from the target device. The communication logic may be configured to instruct a drone to carry a package to the target device in response to the request signal. The drone may be configured to perform an environmental scan during transit to adjust a route to the target device. Other embodiments may be disclosed and/or claimed.

### CROSS-REFERENCE TO RELATED APPLICATION

This application claims priority to U.S. Provisional Patent Application No. 61/813,068, filed Apr. 17, 2013, entitled “SYSTEMS AND METHODS FOR PROVIDING NEUROPROTECTIVE STIMULI,” the entirety of which is hereby incorporated by reference herein.

### BACKGROUND

State-of-the-art approaches to a number of challenges that arise on the battlefield, in hospitals, and in time-sensitive delivery settings may leave much to be desired. For example, the number of cases of head trauma in the United States is tremendous; as reported by Lance Madden in Forbes Magazine Online on Jul. 16, 2012, “[i]n a 2000 study conducted by the American Academy of Neurology, 60 percent of NFL players said they have suffered concussions in their career, and about a third of those players reported having three or more. The US military has reported almost 230,000 cases of traumatic brain injury among more than 2 million Americans who have been deployed to Iraq and Afghanistan.” Some studies have examined the effects of releasing agents (e.g., methamphetamine), which may increase extracellular concentrations of neurotransmitters (e.g., serotonin (5-HT), norepinephrine and dopamine), when these agents are given after traumatic brain injury or an event that deprives cells of oxygen or glucose. For example, researchers at Montana State University have claimed that brain cell damage decreases when methamphetamine is given timely to a victim after a brain trauma event. In U.S. Patent Publication No. 2011/010562, these researchers purport to have obtained results that indicate that serotonin produced a moderate neuroprotective response, norepinephrine also produced a moderate neuroprotective response, and in contrast, dopamine induced a potent dose-dependent neuroprotective response at all concentrations tested. However, methamphetamine is a controlled substance known to be addictive with a high potential for abuse. Consequently, existing approaches for dealing with head trauma may be inadequate.

Other technologies that are emerging in military and civilian contexts also present challenges. In part to reduce the risk to human life and health, drone delivery systems have been identified as a promising technology in military applications, as well as in civilian applications (e.g., commercial delivery). However, these systems have not been designed for robust deployment in changing environments. Additionally, medical interventions after a disfiguring accident (e.g., surgical reconstruction) are still typically performed manually with little guidance to the medical professional, and thus have also not been designed with the ability to readily achieve custom contours to properly reconstruct a patient's body.

### BRIEF DESCRIPTION OF THE DRAWINGS

FIG. 1 is a block diagram of a neuroprotection system, in accordance with some embodiments.

FIG. 2 is a block diagram of a condition detection system that may be included in a neuroprotection system, in accordance with some embodiments.

FIG. 3 is a block diagram of a delivery system that may be included in a neuroprotection system, in accordance with some embodiments.

FIG. 4 is a block diagram of a remote device that may be included in a neuroprotection system, in accordance with some embodiments.

### DETAILED DESCRIPTION

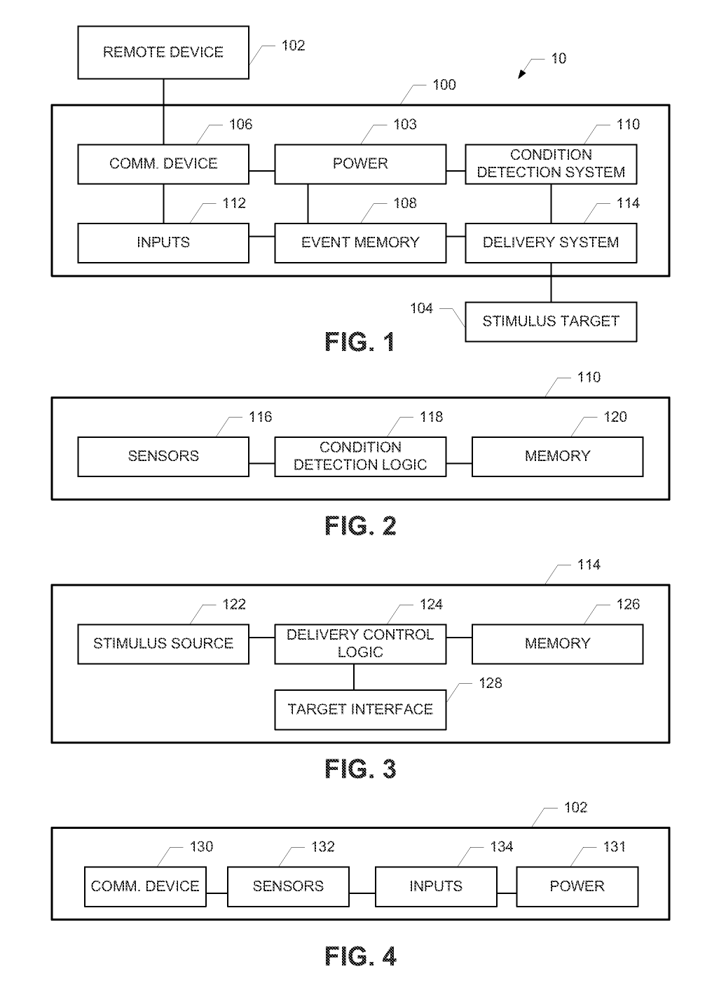

FIG. 1 is a block diagram of a neuroprotection system 10. Various embodiments of neuroprotection systems and methods described herein, such as the neuroprotection system 10, may act to reduce or limit the damage to cells of a target individual (herein referred to as the “target”) caused by trauma or other conditions (herein generally referred to as a “damage condition” or “damage event”) by timely supplying a stimulus to the target's nervous system to increase the production of neuroprotective compounds. In some embodiments, the neuroprotection system 10 may be applied by medical personnel in a treatment center or in proximity to a treatment center (e.g., after a sports injury or car accident), by the target him/herself, or automatically in response to detection of a damage condition. In some embodiments, the neuroprotection system 10 may be used in combat situations. In some embodiments, the stimulus may include a drug stimulus, an electrical stimulus, or both.

Various embodiments of the neuroprotection systems and methods disclosed herein may overcome one or more of the barriers that have impeded the development of effective neuroprotective technology, such as the hesitation of parents to give their children doses of potentially addictive substances, the challenges of custody controls of such substances, transportation and logistic distance and time delays in delivery (e.g., the variability of helicopter delivery schedules due to weather and other challenges of remote locations). Embodiments in which the stimulus is an electrical stimulus may reduce or eliminate the challenges of drug security and delivery, and provide a readily reusable and secure system. Additionally, the systems and methods described herein may be used, in some embodiments, to provide smaller levels of stimulus to targets as a preventative measure to provide neuroprotection during higher risk activities that could result in brain damage.

Although various components of FIG. 1 are indicated by solid lines as being communicatively or otherwise coupled, any one or more components of FIG. 1 may be communicatively or otherwise coupled as suitable to implement the mechanisms described herein. The neuroprotection system 10 may include a local device 100 and a remote device 102. Although only one local device 100 and one remote device 102 are depicted in FIG. 1, the neuroprotection system 10 may include any number of local devices and/or remote devices. In some embodiments, the neuroprotection system 10 may not include any remote devices. The local device 100 may include a power source 103. The power source 103 may include one or more batteries or other power storage devices, one or more solar cells or other power generation devices, one or more transformers that is configured to receive power from an external source (e.g., via induction or by a direct coupling with a source of AC or DC power), or any other suitable power source.

The local device 100 may include a communication device 106, which may provide wired and/or wireless communication capabilities between the local device 100 and the remote device 102. The communication device 106 may provide wired and/or wireless communication capabilities between the local device 100 and one or more additional local devices instead of or in addition to wired and/or wireless communication capabilities between the local device 100 and the remote device 102. In some embodiments, the communication device 106 may be configured to communicate with a computing network, such as the internet, an intranet, or a wireless device network (e.g., a mesh network). Other examples of communication protocols that may be used include frequency-based wireless communication, time-based wireless communication, amplitude-based wireless communication, near-field communication, laser communication (e.g., laser spread spectrum), digital spread spectrum, one way protocols, two way protocols, location-based protocols, and/or a combination of these or any other suitable protocol.

In some embodiments, commands may be transmitted to the local device 100 via the communication device 106 to turn on the device (e.g., by transmitting appropriate control signals to the power source 103), and/or place the local device 100 in a standby or other mode (which may also include control signals transmitted to the power source 103). Such commands from a network may be triggered based on data from sensors local to the local device 100, sensors remote from the local device 100, instructions provided to computing device connected to the network by a person who has access rights to issue commands to the local device 100 (e.g., a medic or other technician at a remote monitoring station).

As used herein, references to “a sensor” or “sensors” may include any one or more sensor fusion packages. A sensor fusion package may include multiple sensors that generate sensed data from disparate sources and/or stored data sources, and that combine the data to provide one or more composite data sets. For example, references to “LIDAR” may include LIDAR fusion packages in which LIDAR data is combined with stored data and/or data sensed by other sensors to provide one or more composite data sets. This stored data and/or sensed data may include, for example, Global Positioning System (GPS) data, satellite images, or visible light images. “LIDAR” may include any one or more suitable frequency ranges (e.g., ultraviolet, visible and/or near-infrared). For example, 3D LIDAR may use temporal frequency signatures that may cover various spectrums (e.g., from infrared to X-ray).

In some embodiments, the communication device 106 may try to communicate with the network upon power-up, and if no connection is established, the local device 100 may enter an autonomous mode in which programmed instructions stored in the memory 108 (discussed below) may be executed. Autonomous mode may include time constraints, stimulus dose constraints, and/or location-based constraints, for example.

The following examples illustrate some of the constraints that may be used, alone or in combination; these examples are purely illustrative, and not limiting. In some embodiments, a location-based constraint may specify that, in an area in which help is typically available nearby (e.g., in the United States in some embodiments) and thus there is less of a need to administer a higher stimulus dose (e.g., in terms of electrotherapy frequency or amperage), the local device 100 may be turned off or certain features may be disabled (e.g., software features). The local device 100 may be configured with information about different location-based needs for neuroprotection and/or cranial stimulation for pain relief. In some embodiments, the local device 100 may be configured to only allow a stimulus to be applied for a certain amount of time or up to a maximum dose over a particular period of time. For example, the local device may be configured for use up to 20 minutes per day, with 2 milliamps delivered via a certain electrode configuration. Further, in some embodiments, this program may only be delivered when the local device 100 is within a specified area (established by, e.g., a geofence). A soldier who has pain, for example, may be able to release cranium-stimulated beta-endorphin for ten minutes every six hours for a specified number of days.

In some embodiments, an autonomous mode of the local device 100 is configured such that, when sensors register an event of overpressure and/or high accelerometer readings (and/or combined with other sensor data), the local device 100 may follow an automatic protocol of administering a programmed dose. For example, the local device 100 may deliver 2 milliamps of stimulus along with a specified amount of a releasing agent and/or other medicine. Before, during, or after the delivery of the programmed dose, the local device 100 may try to communicate with the network to upload information about the event and await additional instructions. If no communication is established (e.g., within a specified time window or windows), the dose may stay at 2 milliamps for twenty minutes every hour; if communication is established, the local device 100 may change the dose (by, for example, increasing milligrams or milliamps for thirty minutes, or adjusting which electrodes or frequencies are being used), in order to maximize treatment benefit. Various autonomous modes may have different settings for different locations and or different users. In some embodiments, a local device 100 configured for an elite team may have relatively few restraints compared with a local device 100 configured for a team with less training. Constraints may also vary by individual user.

In some embodiments, electrodes included with the local device 100 may be used to distinguish users and thus their user and control rights. In some embodiments, different electrical signatures of different users may serve as login and/or authentication data to activate various aspects of the local device 100. These electrodes may be the same as or different from electrodes used to deliver cranial stimulation. For example, in some embodiments, the electrodes may be located in gloves or on the local device 100. Similar use constraints may be applied to weapons, vehicles, aircraft, rocket launchers, etc., in accordance with the systems and techniques disclosed herein.

The local device 100 or the network may be configured with overrides based on movement of the target and/or biomedical information. In some embodiments, an override may be triggered via a command from a network. Such embodiments may be particularly useful when a target or hostile entity is dynamic. For example, if the network knows that the hostile entity is moving towards a target (equipped with the local device 100) at a particular speed (e.g., measured in miles per hour), the network may issue commands to change the geofence based on the movement information and may activate or deactivate different programming on the local device 100. This may be for neuroprotection or to increase focus or other desired effects. The local device 100 may then send back information from each individual user's sensors. In another example, a drone, satellite or other device may use sensors (e.g., Flash, 3D, LIDAR) to detect a shell and then calculate its trajectory towards friendly entities. The device may use a network or other communication protocol to send signals to automatically begin delivery of a stimulus dose or other programming before impact to decrease negative impact to brain cells. The local device 100 may be configured to respond in other ways to a command from a network or other device (e.g., to drop a face shield).

In some embodiments, a person with appropriate rights could disable or change a stimulus dose manually. For example, a medic may have rights to manual override on certain features. Such embodiments may be particularly useful, for example, when a group has lost communication or has purposely turned off all communication in order to avoid being detected. The medic may decide to increase, decrease, or otherwise change a programmed stimulus dose based on the medic's rights.

In some embodiments, if the local device 100 or another part of the neuroprotection system 10 is believed to have been compromised, the person with the locally highest administrative rights may turn off certain network rights to local devices, and/or the person may or may not decide to keep local network control or switch off all features.

In some embodiments, location-based rights may be implemented in a separate subsystem of the local device 100. Location-based rights may compare a magnetometer reading with location information to see if they match. This check may be useful in environments in which location spoofing is a concern as an enemy countermeasure, or when an enemy may try to use the local device 100 in a friendly area by spoofing. Double custody control of various features and/or operations of the local device 100 may be used. In some embodiments, if a network or network operator determines that the network may have been compromised, a person with network rights could turn the system 10 off, or a manual opt-in system could be used. If the network or a network operator has determined that the system 10 may have been hacked, the network may send off a signal or other protocol to all local devices 100. A local device 100 could also have a built in failsafe to never administer over a specified amount of stimulus dose, regardless of instructions to the contrary.

The local device 100 may include one or more inputs 112. The inputs 112 may include any of a number of devices that allow the local device 100 to receive inputs, such as one or more buttons, key pads, touch pads, dials, proximity sensors (e.g., radio frequency identification sensors), key/lock mechanisms, bar code or other code readers (such as quick response (QR) code readers), cameras, and/or microphones, for example. In some embodiments, the local device 100 may be turned on manually with various choices of settings. In some embodiments, a setting may include a program menu of treatment options. The programs available may be limited based on sensor data. More options may be available if one or more sensors registered an event. In some embodiments, settings may be based on a soldier's weight, last dose amount, time since last dose, and/or any other suitable information. In some embodiments, the local device 100 may store this data, or the user may carry a data storage device that communicates this information to the local device 100. Manual settings may include amount of dose, time of delivery, self-dosing restrictions, frequency, location of electrodes, milliamps of electrical stimulation, and/or other suitable settings. In some embodiments, a setting may be as simple as 3 milliamps or 4 milliamps with an auto timer. The settings could also include number of occurrences and time between doses.

The local device 100 may include a memory 108. The memory 108 may include any one or more data storage devices, such as RAM, Flash memory, or solid state memory. In some embodiments, the memory 108 may store status information about the local device 100 (e.g., records of power on/power off, stimulus delivery data, sensor data, and/or hardware/firmware/software version data). In some embodiments, the memory 108 may store biomedical or other information related to one or more targets with whom the local device 100 is associated. This information may be programmed into the memory 108 by a user input device (e.g., a keyboard or touchpad), via a network connection, or may be selected from a list or dial setting. The memory 108 may be configured to store any of the information described herein as stored or accessed by the local device 100.

The local device 100 may include a condition detection system 110. The condition detection system 110 may be configured to determine whether a neuroprotective stimulus should be delivered to a stimulus target 104 (e.g., a human or animal) based at least in part on one or more conditions detected in the target's environment or person. In some embodiments, when one or more sensors or inputs of the condition detection system 110 (and/or one or more sensors or inputs of the remove device 102) indicate that a traumatic brain injury is likely, or potentially likely, the condition detection 110 may generate control signals and transmit those control signals to a delivery system (such as delivery system 114, discussed below), which may respond by administering a stimulus program.

The local device 100 may include a delivery system 114. The delivery system 114 may be configured to deliver a neuroprotective stimulus (e.g., an electrical stimulus and/or a drug stimulus) to the stimulus target 104 in response to the detection of appropriate conditions by the condition detection system 110.

FIG. 2 is a block diagram of an embodiment of the condition detection system 110 of the neuroprotection system 100 of FIG. 1. Although various components of FIG. 2 are indicated by solid lines as being communicatively or otherwise coupled, any one or more components of FIG. 2 may be communicatively or otherwise coupled as suitable to implement the mechanisms described herein.

The condition detection system 110 may include one or more sensors 116. In some embodiments, one or more of the sensors 116 may include a sensor designed for inclusion in a helmet, headband, eyewear, or other wearable item to measure traumatic force to a wearer of the item. For example, one or more of the sensors 116 may include a sensor designed for the U.S. military's “Advanced Combat Helmet” program. One such sensor is the “Headborne Energy Analysis and Diagnostic Systems (HEADS)” manufactured by BAE Systems of the United Kingdom. As reported by Lance Madden in Forbes Magazine Online on Jul. 16, 2012, “[when] [p]laced inside of a soldier's helmet and weighing just 2 ounces, the [HEADS] sensor collects data of hits from explosive devises and other blunt impacts, including impact location, magnitude, duration, blast pressure, ambient temperature and the exact times of impacts. The NFL wants to use the same sensors, though altered slightly, in football helmets to track the severity of blows to the head, so players may be taken out of games before severe brain trauma—namely concussions—occurs or escalates.”

In some embodiments, one or more of the sensors 116 may be used to monitor the target (e.g., temperature, acceleration, heart rate, blood pressure, oxygen saturation, etc.). This data may be used by the delivery system 114 (discussed below) to adjust the stimulus program delivered to the target. Thus, in some embodiments, treatment and feedback may occur simultaneously or substantially simultaneously.

The condition detection system 110 may include condition detection logic 118. The condition detection logic 118 may include any one or more processors, special purpose computing chips, logic devices, or other computational devices. The condition detection logic 118 may include local or onboard decision software and/or may communicate with condition detection logic implemented at a network or other remote device (such as the remote device 102). In some embodiments, the condition detection logic 118 may implement a decision tree based on one or more factors or factor weights, which may include (but are not limited to) distance and/or time to treatment, location of the target or the local device 100, location of others or devices, sensor reading(s), shock wave or overpressure information, impact severity information, whether a medic is stationed within a target's unit, whether a treatment center is in close proximity, etc.

The condition detection system 110 may include a memory 120. The memory 120 may include any one or more data storage devices, such as RAM, Flash memory, or solid state memory. In some embodiments, the memory 120 may store biomedical or other information related to one or more targets with whom the local device 100 is associated. This information may be programmed into the memory 120 by a user input device (e.g., a keyboard or touchpad), via a network connection, or may be selected from a list or dial setting. The condition detection logic 118 may use the information about a target stored in the memory 120 in determining whether a damage event has occurred (e.g., using a target's weight to determine acceleration).

In some embodiments, signals generated by the condition detection system 110 may be communicated to remote devices via the communication device 106 of the local device 100. For example, in some embodiments, a drone in standby may automatically launch a medicine drop or redirect a medicine drop or otherwise engage in communication based on signals generated by the condition detection system 110 (indicating, for example, that a damage event has occurred).

In some example scenarios, an event may be registered by sensors or an indication of an event may be received via a communication channel. The event may have been registered in an area where there are assets or other entities of interest. The event may be associated with a high likelihood of rescue vehicle being deployed (e.g. based on distance from treatment, area being crossed to get to event, hostile activity, etc.). A computer or person may calculate the route(s) that a helicopter or other vehicle would take to reach the target. A drone may be launched or redirected to the target, and may scan the route for signatures of possible hostiles. Examples of signatures may include vehicles with certain characteristics, new items on roadway that were not there previously, cell phones that are on or recently turned off within time vectors of route, back scatter that correlates to vehicles that are not historically in the area, unusual radio frequencies, vehicles, people, traffic, etc. The drone (or other sensor-bearing vehicle) may scan an area for a suitable landing zone (which may include taking or referencing LIDAR readings of the land surface). The drone may fly the route in front of the rescue helicopter giving advance warning and/or may have time to fly alternate routes, depending on scan information or known problems with a route. In some embodiments, the drone may be at a high enough location to scan many routes in detail. In some embodiments, several drones may be at different locations to decrease time to cover the scanning. Some drones may be configured with signatures to draw out hostiles before a rescue helicopter follows route. Such signatures may include a drone emitting different frequencies in different patterns to simulate a rescue helicopter or other aircraft to draw out enemy information. In some embodiments, a drone may scan the ground on a roadway that a rescue vehicle/rescue drone vehicle may follow. The drone may be looking for improvised explosive devices (IEDs) that may be buried or placed roadside. The drone may be configured to compare historical fly over data with current or more recent drone flyovers to determine whether the road surface was disturbed or elevated/depressed by digging. For example, backscatter may show a difference in elevation and composition of spin of disturbed earth. A drone may determine a landing zone based on this data and elevation of ground and/or density. An aircraft may then land and deploy a drone rescue vehicle that follows a route partially based on the scanned route on the ground. This may result in decreasing the time to the treatment facility, saving more brain cells and possibly lives.

In some embodiments, an enemy may figure out that drones do flyovers as a precursor to sending out rescue vehicle craft; thus, in some embodiments, separate drones may be deployed to obscure the route and confuse the enemy.

In some embodiments, the same drone or a separate drone may deliver supplies. For example, a drone may perform supply drops based on a predicted need of supplies based on historical needs and current use of supplies. Newly requested or high priority supplies may override or change the payload or delivery schedule. A drone, like an automated warehouse, may be configured to simply fill its containers based on priority need and make an air drop or land delivery. This may allow teams to travel lighter and receive medical supplies quickly. One or more computing devices (e.g., the local device 100, a drone, and/or a networked device) may monitor the rate of usage of supplies and decide that sufficient supplies remain for a certain period of time (e.g., five days) but that a smaller weather window is available in that period (e.g., three days out of the next six), so the computing device may determine that an early drop is to be performed. The computing device may also predict that a warehouse may need additional supplies.

In some embodiments, signals generated by the condition detection system 110 (such as alerts) may be transmitted to remote devices (e.g., helicopters or drone rescue vehicles) to trigger the power on of the remote devices, thereby decreasing the response time of those remote devices. In some embodiments, a remote device such as a drone may or may not try to establish connection with the local device 100 if, for example, a wireless signal generated by the communication device 106 is low. In some embodiments, a remote device such as a drone may operate as a repeater for signals generated by the local device 100.

FIG. 3 is a block diagram of an embodiment of the delivery system 114 of the neuroprotection system 100 of FIG. 1. Although various components of FIG. 3 are indicated by solid lines as being communicatively or otherwise coupled, any one or more components of FIG. 3 may be communicatively or otherwise coupled as suitable to implement the mechanisms described herein.

In some embodiments, the delivery control system 114 (and potentially other components of the local device 100) may be incorporated into a combat or athletic helmet, or a garment to be worn under a helmet. The delivery control system 114 may be permanently secured to the helmet or may be temporarily or removably secured. In some embodiments, the delivery control system 114 may only operate correctly when it is secured to a helmet or other wearable item; in other embodiments, the delivery control system 114 may operate correctly when it is detached from a helmet or other wearable item to which the delivery control system 114 was previously coupled. In some embodiments, the wearable item with which the delivery control system 114 is coupled (e.g., clothing or a helmet) may include an inner layer of blood clotting powder/material that may enter a wound along with any debris fragments to aid in clotting of the wound.

The delivery control system 114 may include a stimulus source 122. In some embodiments, the stimulus source 122 may include a supply of a drug that may be delivered to the target under the control of the delivery control logic 124. The drug may be a releasing agent, such as amphetamine or methamphetamine or another releasing agent or combination of releasing agents, which may increase the extracellular concentration of neurotransmitters (such as serotonin, norepinephrine and/or dopamine). In some embodiments, the stimulus source 122 may include wet and/or dry ingredients that are mixed automatically when a damage event is detected or upon another suitable condition (e.g., the location of the target, the availability of a network communication signal, or the unavailability of a network communication signal). In some embodiments, when an event is detected, an intravenous (IV) pump may be unlocked and ready for use. In some embodiments, the stimulus source 122 may include a pump for oral dispensing or other types of dispensing. In some embodiments, the stimulus source 122 may include a dry powder that is mixed with a fluid to form a fluidized stimulus drug, which may then be delivered to the target (and possibly additional targets)

In some embodiments, the stimulus source 122 may include an electrical power supply (e.g., a battery) that may be used to generate an electrical stimulus (e.g., a current stimulus) for delivery to the target. This electrical stimulus may be delivered instead of, or in addition to, a drug stimulus. Delivery of appropriate current stimuli may increase the level of dopamine in a target's brain, which may serve a neuroprotective function. In some embodiments, delivery of appropriate cranial stimulation may increase the effective time of natural or (RA) dopamine in a target's brain which may serve a neuroprotective function and potentially release additional dopamine. Examples of studies describing stimuli that may be used in the systems and methods described herein are presented below.

Embodiments of the delivery system 114 in which an electrical stimulus is used in lieu of a drug stimulus may be advantageous in avoiding the use of potentially addictive drugs (e.g., methamphetamine), especially when used during “higher risk” activities in which a target is likely to be repeatedly dosed (e.g., activities in which the target is exposed to overpressure, shockwaves, or impacts). In other words, embodiments using an electrical stimulus may be more frequently used without risk of drug addiction than embodiments using a drug stimulus. Embodiments of the delivery system 114 in which an electrical stimulus is used in lieu of a drug stimulus may also be more readily reusable and may have a smaller form factor (which may enable the neuroprotection system 10 to fit into a helmet, or a layer beneath a helmet, and be connected with the condition logic 118 and/or one or more sensors 116 of the condition detection system 110).

In some embodiments, the electrical stimulus may follow a transcranial direct current stimulation (TDCS) protocol. One example of such a protocol includes the delivery of two milliamperes of direct current for 30 minutes through electrodes applied to the scalp of the target. TDCS protocols that may be used have been developed by the Air Force Research Laboratory and were described at the annual meeting of the Society for Neuroscience on Nov. 13, 2011. The Air Force Research Laboratory protocols are reported to have improved the robustness and organization of bundles of nerve fibers below the brain's surface as early as five days after the application of the protocol. A TDCS protocol may be applied as a preventative measure, on an intermittent or periodic basis, to improve a target's resistance to traumatic brain damage. Other examples of electrical stimulus programs that may be delivered by the delivery system 114 include transcranial direct current stimulation, transcranial magnetic stimulation (TMS), repetitive TMS, single pulse TMS, transcranial random noise stimulation (TRNS), transcranial alternating current stimulation (TACS), transcranial high frequency stimulation, and transcranial pulsed ultrasound.

The delivery control system 114 may include delivery control logic 124. The delivery control logic 124 may include any one or more processors, special purpose computing chips, logic devices, or other computational devices. In some embodiments, the delivery control logic 124 may be configured to select a stimulus program from a set of predetermined stimulus programs or based on a stimulus program-generation routine. The stimulus program selected may depend on the damage condition detected by the condition detection system 110. Parameters that may vary between different stimulus programs may include milliamps of current delivered, time of delivery, frequency of delivered pulses, and the location of stimulation, for example. The stimulus programs may be configured to expire after a certain amount of time has passed or a particular date has been reached, which may be configured by a local or remote administrator. The ability of the delivery system 114 to deliver stimulus may also be based on sensor requirements and/or geofence requirements.

Several studies may be cited to support the potential for the stimuli described herein to increase dopamine. One mechanism may include prolonging the after effects of dopamine. The source of dopamine could be from an individual's own body, which may naturally release dopamine, or from the introduction of a releasing agent, or both. The brain may release dopamine from doing something that excites the individual.

The systems and methods disclosed herein may include one or more of several mechanisms for neuroprotection. In accordance with some mechanisms, less methamphetamine (RA) with stimulation may provide neuroprotection. In accordance with some mechanisms, cranial stimulation and getting excited about something (when excited, more natural dopamine is produced) may provide neuroprotection. In accordance with some mechanisms, if the subject about which an individual is learning is not exciting to him or her, a little releasing agent and cranial stimulation may improve memory. In accordance with some mechanisms, less methamphetamine may be desirable for avoiding negative side effects, especially if one wants to build up neuroprotection over a long time period. In accordance with some mechanisms, the systems and methods disclosed herein may be beneficial in reducing the amount of attention-deficit/hyperactivity disorder (ADHD) RA's given to children.

As described by Kuo et al. in “Boosting focally-induced brain plasticity by dopamine” (Cereb. Cortex (2008) 18 (3): 648-651), regarding dopamine and transcranial direct current stimulation (tDCS), “administering L-dopa turns the unspecific excitability enhancement caused by anodal tDCS into inhibition and prolongs the cathodal tDCS-induced excitability diminution. Conversely, it stabilizes the [paired associative stimulation] PAS-induced synapse-specific excitability increase. Most importantly, it prolongs all of these aftereffects by a factor of about 20. Hereby, DA focuses synapse-specific excitability-enhancing neuroplasticity in human cortical networks.”

As described by Li et al. in “Anodal transcranial direct current stimulation relieves the unilateral bias of a rat model of Parkinson's disease,” (33rd Annual International Conference of the IEEE EMBS, Boston, Mass. USA, Aug. 30-Sep. 3, 2011, p. 767), regarding increasing dopamine, “In addition, tDCS may cause dopamine release in the caudate nucleus or in the striatum as [repetitive transcranial magnetic stimulation] rTMS does. Because the dopaminergic action maybe reflected by the prolonged cortical silent period, and tDCS on M1 could prolong the cortical silent period which is associated with the excitability of the motor cortex.” See also A. P. Strafella, T. Paus, J. Barrett, and A. Dagher, “Repetitive transcranial magnetic stimulation of the human prefrontal cortex induces dopamine release in the caudate nucleus,” J Neurosci, vol. 21, pp. RC157, August 2001, and A. P. Strafella, T. Paus, M. Fraraccio, and A. Dagher, “Strital dopamine release induced by repetitive transcranial magnetic stimulation of the human motor cortex,” Brain, vol. 126, pp. 2609-2615, December 2003.

As described by Kamida et al. in “Transcranial direct current stimulation decreases convulsions and spatial memory deficits following pilocarpine-induced status epilepticus in immature rats” (Behavioural Brain Research, Volume 217, Issue 1, 2 Feb. 2011, Pages 99-103), “[t]hese findings suggested that cathodal tDCS has neuroprotective effects on the immature rat hippocampus after pilocarpine-induced [status epilepticus] SE, including reduced sprouting and subsequent improvements in cognitive performance.”

As described by DosSantos et al. in “Immediate effects of tDCS on the u-opioid system of a chronic pain patient” (Front. Psychiatry, 3:93, 2012), “[i]nterestingly, the single active tDCS application considerably decreased [u-opioid receptor non-displaceable binding potential] pORBP_{ND} levels in (sub)cortical pain-matrix structures compared to sham tDCS, especially in the posterior thalamus. Suggesting that the p-opioidergic effects of a single tDCS session are subclinical at immediate level.”

In some embodiments, sensors may include biomedical vitals, body temperature, blood pressure, pulse oxygen sensors, etc. Certain programs may be limited to certain areas or zones when, for example, a group using the system 10 may only want certain features used in certain areas or under certain conditions. For example, the military may want to deliver, to a soldier who just was injured, a dose of electrically stimulated beta-endorphin (a natural and very strong painkiller). This delivery may take place in autonomous mode or an offline mode, and may be based on data from one or more biomedical sensors or medic rights to the device. These rights may change based on location. A unit that is approaching a firefight may want to pre-dose to fight through the high likelihood of distracting pain. The most suitable programs and operations may be determined on a case by case basis. In some situations, pain may serve a positive purpose, while in others, pain may prevent a positive response (e.g., preventing one from getting out of harm's way, or stopping a person from crawling down a mountain). In various embodiments, the delivery control logic 124 may or may not be configured to deliver maintenance stimulus doses to the target, based on sensor data, networked instructions, manual inputs or failsafe overrides, for example.

The delivery control system 114 may include a memory 126. The memory 126 may include any one or more data storage devices, such as RAM, Flash memory, or solid state memory. In some embodiments, the memory 126 may store biomedical or other information related to one or more targets with whom the local device 100 is associated. This information may be programmed into the memory 126 by a user input device (e.g., a keyboard or touchpad), via a network connection, or may be selected from a list or dial setting. The delivery control logic 124 may use the information about a target stored in the memory 126 in determining an appropriate stimulus program (e.g., using a target's weight or age to set stimulus parameters).

The delivery control system 114 may include a target interface 128. The target interface 128 may be configured to contact or otherwise interface with the tissue of the target to supply a stimulus under the control of the delivery control logic 124. In some embodiments, the target interface 128 may include a personal jet injector that may be worn next to a target's skin to automatically deliver a drug in accordance with instructions from the delivery control logic 124. A jet injector may also be unlocked for use on multiple patients. In some embodiments, the target interface 128 may include one or more electrodes, which may include integrated conductive gel layers or conductive pins for contact with the target's skin. In some embodiments, the target interface 128 may include one or more dry electrodes that have comb-like features that can penetrate hair to contact the target's scalp. In some embodiments, a conductive gel or other material could be released or secreted in response to a damage event. In some embodiments, springs or a light pressure device may release in response to a damage event to help with conductivity or connection between the delivery system 114 and the target. In some embodiments, an array of electrodes may be placed through the helmet, garment or other wearable item. One or more of these electrodes may be activated at different times in response to various conditions (e.g., in accordance with the stimulus program or based on alignment of the target's head after a damage event).

Electrodes may also be selectively turned on based on connectivity for redundancy. For example, electrodes may be changed when the local device 100 is programmed to determine that another electrode is in close proximity and would provide the same effect, but has less resistance (e.g., better conductivity). The local device 100 may be configured to raise or lower output based on resistance to deliver an accurate and constant dose.

In some embodiments of the local device 100, a helmet or electrode cap may include a few electrodes, over a hundred electrodes, or any number of electrodes. As the number of electrodes increases, the inter-electrode distance decreases. Electrodes near each other may be able to work together or work as backups. If one electrode encounters too much resistance or has a fault, software programmed into the local device 100 may control a switch to an electrode that does not.

In some embodiments, the local device 100 may include many small electrodes covering the cranium in different areas. Different treatments may use different cranial sites or electrodes. In some embodiments, if the electrodes are clustered close enough, they may be used as a redundant system. The programming and type of treatment would dictate which electrodes or sites are used and may be part of an automated stimulus delivery program. As technology miniaturizes electrodes, the number of electrodes is increased, and flexible electrode sheets become available, the number of electrode sites may increase.

An example program that may use different electrode locations for pain management is described in Aarts et al. in “Treatment of ischemic brain damage by perturbing NMDA receptor-PSD-95 protein interactions” (Science 298(5594): 846-50, 2002). According to Aarts et al., “N-methyl-D-aspartate receptors (NMDARs) mediate ischemic brain damage but also mediate essential neuronal excitation. To treat stroke without blocking NMDARs, we transduced neurons with peptides that disrupted the interaction of NMDARs with the postsynaptic density protein PSD-95. This procedure dissociated NMDARs from downstream neurotoxic signaling without blocking synaptic activity or calcium influx. The peptides, when applied either before or 1 hour after an insult, protected cultured neurons from excitotoxicity, reduced focal ischemic brain damage in rats, and improved their neurological function. This approach circumvents the negative consequences associated with blocking NMDARs and may constitute a practical stroke therapy.”

Another program that may be initiated would be a mix of PSD-95 and releasing agent and cranial stimulation. This may enhance dopamine quickly. The PSD-95 may reduce neurotoxicity without blocking helpful synaptic activity. In some embodiments, there may be a time delay between the delivery of a drug stimulus and the delivery of an electrical stimulus. For example, in some embodiments, an injector or other drug delivery device of the local device 100 may release PSD-95 or dopamine at the time of an event, and the local device 100 may begin to deliver an electrical cranial stimulus after some time period has elapsed from injection/administration. This may be implemented by programming in the local device 100 or by programming in a networked device, and may be based on biomedical and sensor data and/or absorption rate calculations. In some embodiments, it may be beneficial to give a PSD-95 and a time released (RA) for some types of traumatic brain injuries. In some embodiments, it may be advantageous to have PSD-95 absorbed by a user to block NMDA before electrical stimulation begins. Other medicines or treatments in combination may be used.

In some embodiments, one or more of the electrodes used for the delivery of electrical stimulus may also be used to record data from the target (e.g., electroencephalograph (EEG) data or electromyograph (EMG)). The delivery control logic 124 may use this data to adjust the stimulus program delivered to the target. In some embodiments, the recorded data may be stored in the memory 126 and later downloaded from the local device 100 or transmitted to a remote device from the local device 100 (e.g., via a network). A remote device may use the data to instruct the delivery system 114 to change the stimulus program delivered to the target.

FIG. 4 is a block diagram of an embodiment of the remote device 102 of the neuroprotection system 100 of FIG. 1. Although various components of FIG. 3 are indicated by solid lines as being communicatively or otherwise coupled, any one or more components of FIG. 3 may be communicatively or otherwise coupled as suitable to implement the mechanisms described herein. In some embodiments, the remote device 102 may be included in a drone, airborne device, or ground-based or below-ground vehicle or device, for example.

The remote device 102 may include a power source 131. The power source 131 may include one or more batteries or other power storage devices, one or more solar cells or other power generation devices, one or more transformers that is configured to receive power from an external source (e.g., via induction or by a direct coupling with a source of AC or DC power), or any other suitable power source.

The remote device 102 may include a communication device 130, which may provide wired and/or wireless communication capabilities between the remote device 102 and the local device 100. The communication device 130 may provide wired and/or wireless communication capabilities between the remote device 102 and one or more additional local devices instead of or in addition to wired and/or wireless communication capabilities between the local device 100 and the remote device 102.

The remote device 102 may include one or more sensors 132. The sensors 132 may include any of the sensors discussed above with reference to the sensors 116 of the condition detection system 110. The sensors 132 may also include sensors for LIDAR, 3D LIDAR, Flash LIDAR or other sensors that measure pressure or shock waves. In some embodiments, data from one or more of the sensors 132 may be communicated to the local device 100 via the communication device 130 as a signal that may unlock or initiate a stimulus delivery program at the local device 100. For example, a drone or airborne embodiment of the remote device 102 may send a signal to the local device 100 to trigger the powering up of a helicopter or rescue unit based on a damage event.

In some embodiments, data received at the remove device 102 from one or more of the sensors 132 and/or the inputs 134 may be used to turn the local device 100 one or off. Manual on/off control of the local device 100 from the remote device 102 may be advantageous in a number of situations. For example, a person who disables bombs or IEDs may want to turn the local device 100 on manually if he or she feels it helps them concentrate or problem solve better. This may also be the case for a sniper. A SEAL team member may want to turn on the pain reduction program to get over a wall and to safety.

Manual control may be based on trust of the person to whom the local device 100 is issued. For example, a medic in a SEAL may be trusted enough to be able to use manual on off controls (e.g., after training). There may be a need to eliminate all communication signatures so there would not be a network link, which may make manual control advantageous. In some such embodiments, everyone on a team may be able to be trusted and trained. Restrictions may be put in place to prevent misuse or accidental misuse.

There may be scenarios where a manual on would be advantageous. In some embodiments, the local device 100 may be configured to deliver different stimulus programs depending on whether the local device 100 was turned on manually; for example, stimulus programs delivered upon manual turn-on may deliver a lower number of milliamps of stimulus current, or may deliver current over a shorter time duration, than when the local device 100 was turned on automatically when a damage condition was sensed.

The remote device 102 may include one or more inputs 134. The inputs 134 may include any of a number of devices that allow the remote device 102 to receive inputs, such as one or more buttons, key pads, touch pads, dials, proximity sensors (e.g., radio frequency identification sensors), key/lock mechanisms, bar code or other code readers (such as quick response (QR) code readers), cameras, and/or microphones, for example.

In some embodiments, a remote device (such as remote device 102) may include logic and memory configured to allow the remote device to start back tracking data in time to a location of a damage incident, identify unique signatures of vehicles that were in this area, put identified vehicles or people on a watch list or alert list when these signatures reappear in the data stream. Sensors that may be used to provide input data to such logic may include 3D LIDAR or Flash LIDAR, for example. The logic may be configured to exclude friendly or know signatures.

Some or all of the components of the neuroprotection system 10 may be included in a storage device, cabinet, medical supply chest, or other container that is configured to open or unlock one or more compartments to make the delivery system 114 available and usable. Such embodiments may be useful for keeping drugs and/or electrical stimulus delivery systems secured when used in the field. In some embodiments, the delivery system 114 may be made available based on the detection of overpressure, impact or another event, and/or based on commands transmitted over a network in response to a sensor event. In some embodiments, components of the neuroprotection system 10 may be included in a non-networked or networked lock that may or may not have fail safes in the event of lack of communication.

In some embodiments of the containers disclosed herein, the containers and/or associated locks/actuators may be controlled via software permissions modified by a remote or local network device. These devices could base their permission commands in part or whole on a number of factors, such as distance to treatment, location of a target, sensor readings (e.g., impact severity, etc.), whether there is a medic in unit or close in proximity, and geofencing, for example. In some embodiment, a container may operate as a pill counter, and may activate a particular pump or open a particular lock on a schedule for stimulus delivery once a geofence or other signal is detected.

These containers also may have failsafe operation conditions when network communication is not established. In some embodiments, containers or locks may be set to mechanical override or open with overpressure. In some embodiments, containers or locks may use overpressure and/or acceleration data to disengage a lock.

In some embodiments, containers may be portable devices that may be carried on the person (e.g., of a target or medic). These portable containers may take the form of a pill box, pump, or injector, for example. The containers may communicate inventory or tapering data to a remote device over a network for inventory monitoring. For example, in some embodiments, a radio frequency identification (RFID) tag may be packed in a small Faraday cage. When the container is tampered with (e.g., a package or drawer is opened), the cage may be ripped or broken, activating the RFID tag to communicate. In some embodiments, a security strip or seal may include two RFID tags. When the strip or seal is broken, one RFID tag may break a circuit while leaving the other RFID tag intact. The total resulting signal from the strip or seal would change as a result of the break, signifying that the container was opened. The two RFIS tags could be on the same frequency, or on different frequencies.

In some embodiments, the containers may only receive data without transmitting data to avoid giving off electromagnetic or other information about the location of the container.

Various embodiments of the neuroprotection system and methods disclosed herein may be packaged in different forms for different applications. For example, military leaders may want to let the soldiers have a small dose using one form of the neuroprotection system 10 as a precautionary ramp up for neuroprotection if the leaders believe that the soldiers are facing a high likelihood of combat or exposure to impacts or sudden changes.

The neuroprotection systems and methods described herein may have a number of applications in both military and civilian settings. For example, a coach that sees an impact in a helmet based on a sensor reading may have the helmet begin delivering electrical stimulation on the field before the ambulance arrives or a doctor decides what treatment to proceed with. In another example, a coach may decide to have athletes (e.g., boxers) use the neuroprotection system 10 for neuroprotection based on the likelihood of that sport having impacts. The neuroprotection system 10 may be used to treat a target (e.g., with electrical stimulation) who has been unconscious or has experienced any condition in which the target experienced a lack of oxygen or lack of glucose to brain cells, or a target that is likely to undergo brain swelling or infection after an operation. The neuroprotection system 10 may be integrated with a backpack or other item for remote mountain and backpacking applications, and the delivery system 114 may be configured with protocols specific for injuries likely to be encountered in such settings.

The systems and methods described herein may be configured for use with other drugs or stimulus programs in addition to or instead of neuroprotective stimulus programs. For example, other kinds of medicine or therapeutic electrical stimulation may delivered using the systems and methods disclosed herein (e.g., on other parts of a target's body, and in response to different events).

For example, often in the course of active military duty, explosions or combat leads to facial disfigurement. LIDAR could be used to help facial plastic surgeons. If a person's face was scanned prior to the incident, the scan may provide an accurate map of the face to compare to the current face. This may be helpful on many fronts. In some embodiments, a prosthetic nose, chin, etc., could be made in the exact proportions to the old nose, chin, etc. The prosthetic may be adjusted based on the current scan so that if only a part of the chin is missing, the prosthetic manufacturing equipment would have access to data representing the exact specifications of the missing part. An implant could be made, printed, or shaved to the exact size at the doctor's office. In some embodiments, filler could be used to change the volume of areas of the face to match the old proportions. The LIDAR scans of the face prior to the event may serve an important role in reconstruction.

In some embodiments, a plastic surgeon may use LIDAR to guide filler placement so both sides of the face are symmetrical. The LIDAR could indicate with light shades on the face or dots where the filler is needed. The LIDAR could even change color on the face as the desired result is achieved. The LIDAR could also show the surgeon where the delivery point (e.g., needle point) is under the skin with a separate dot. The surgeon would know where their needle is and see it moving towards the correct location. The cannula may even have a mechanism to deliver the precise amount of filler based on LIDAR measurements. The LIDAR could be adjusted to allow overfill.

In some embodiments, a plastic surgeon may use the technology disclosed herein to achieve a more balanced and symmetrical face, or increase fullness symmetrically. This also could be used in commercial settings using formulas for a balanced/symmetrical/golden rule that could achieve custom looks. This could help produce the desired affects more accurately. A patient could see a more accurate depiction of their result based on LIDAR accuracy. This same technique may be used to give a person a virtual face lift on photos and video conferencing on the fly.

The following paragraphs provide various examples of embodiments disclosed herein. Example 1 is a drone delivery system, including: receiving logic to receive a request signal indicative of a package request event proximate to a target device, wherein the request signal comprises sensor data indicative of conditions proximate to the target device or a request signal transmitted to the receiving device from the target device; and communication logic to instruct a drone to carry a package to the target device in response to the request signal;wherein the drone is to perform an environmental scan during transit to adjust a route to the target device.

Example 2 may include the subject matter of Example 1, and may further specify that the receiving device and the communication device are included in the drone.

Example 3 may include the subject matter of any of Examples 1-2, and may further specify that the package includes a medicine and the request signal is indicative of a damage event.

Example 4 may include the subject matter of Example 3, and may further specify that the medicine is a neuroprotection medicine.

Example 5 may include the subject matter of any of Examples 1-4, and may further specify that perform an environmental scan during transit comprises scan for signatures of possible hostiles.

Example 6 may include the subject matter of any of Examples 1-5, and may further specify that perform an environmental scan during transit comprises scan for a landing zone.

Example 7 may include the subject matter of any of Examples 1-6, and may further specify that the communication logic is further to instruct a second drone to travel ahead of the drone and perform an environmental scan during transit.

Example 8 may include the subject matter of any of Examples 1-7, and may further specify that perform an environmental scan during transit comprises comparing current environmental data with stored historical environmental data.

Example 9 may include the subject matter of any of Examples 1-8, and may further include route determination logic to determine a route for an emergency vehicle to follow to the target device based on the environmental scan.

Example 10 may include the subject matter of any of Examples 1-9, and may further specify that the request signal is generated based on historical use of the package.

Example 11 include the subject matter of Example 10, and may further specify that the request signal is generated by a computing device remote from the target device.

Example 12 is a drone request system, including: condition detection logic to identify a package request event proximate to the drone request system, wherein the package request event is identified based on one or more sensor signals; and transmitting logic to transmit a request signal indicative of the package request event to a drone delivery system, wherein the drone delivery system is to instruct the drone to carry a package to the drone request system in response to the request signal.

Example 13 may include the subject matter of Example 12, and may further specify that the package request event is a damage event.

Example 14 may include the subject matter of any of Examples 12-13, and may further include a delivery system to deliver a medical treatment to a user proximate to the drone request system in response to identification of the package request event.

Example 15 may include the subject matter of Example 14, and may further specify that the delivery system is to deliver the medical treatment to the user in response to a command signal from the drone.

Example 16 is a reconstructive surgery support system, including: a memory storing a LIDAR scan of a portion of a patient's body prior to a damage event affecting a contour of the portion of the patient's body; and a visual indication system to project a visual indicator of a pre-damage contour of the portion of the patient's body, on the portion of the patient's body, based on the LIDAR scan.

Example 17 may include the subject matter of Example 16, and may further specify that the visual indication system includes feedback logic to change the visual indicator as surgical reconstruction is performed on the portion of the patient's body to indicate a difference between the pre-damage contour and a current contour.

Example 18 may include the subject matter of Example 17, and may further specify that change the visual indicator comprises change the color of the visual indicator.

Example 19 may include the subject matter of any of Examples 17-18, and may further specify that the surgical reconstruction comprises the application of a filler.

Example 20 may include the subject matter of any of Examples 16-19, and may further include surgical implement indicator logic to provide a visual indicator of a location of a surgical implement under the patient's skin during surgical reconstruction.

Example 21 may include the subject matter of Example 20, and may further specify that the surgical implement is a point of a needle.

Example 22 may include the subject matter of any of Example 16-21, and may further include filler analysis logic to determine an amount of filler to be delivered to match a current contour of the portion of the patient's body with the pre-damage contour.

Example 23 may include the subject matter of Example 22, further comprising:

filler delivery logic to deliver the determined amount of filler.

Example 24 is a plastic surgery support system, including: a memory storing an image of a desired contour of a portion of a patient's body; a LIDAR system to image a current contour of the portion of the patient's body; and a visual indication system to project a visual indicator of the desired contour of the portion of the patient's body, on the portion of the patient's body, based on the LIDAR system image.

Example 25 may include the subject matter of Example 24, and may further specify that the visual indication system includes feedback logic to change the visual indicator as plastic surgery is performed on the portion of the patient's body to indicate a difference between the current contour and the desired contour.

Example 26 may include the subject matter Example 25, and may further specify that change the visual indicator comprises change the color of the visual indicator.

Example 27 may include the subject matter of any of Examples 25-26, and may further specify that the surgical reconstruction comprises the application of a filler.

Example 28 may include the subject matter of any of Examples 24-27, and may further include symmetry analysis logic to identify asymmetries in the portion of the patient's body and determine the desired contour of the portion of the patient's body to at least partially remedy the asymmetries.

What is claimed is:

1. A drone delivery system, comprising:
receiving logic to receive a request signal indicative of a package request event proximate to a target device, wherein the request signal comprises sensor data indicative of conditions proximate to the target device or a request signal transmitted to the receiving device from the target device; and
communication logic to instruct a drone to carry a package to the target device in response to the request signal;
wherein the drone is to perform an environmental scan during transit to adjust a route to the target device.

2. The drone delivery system of claim 1, wherein the receiving device and the communication device are included in the drone.

3. The drone delivery system of claim 1, wherein the package includes a medicine and the request signal is indicative of a damage event.

4. The drone delivery system of claim 3, wherein the medicine is a neuroprotection medicine.

5. The drone delivery system of claim 1, wherein perform an environmental scan during transit comprises scan for signatures of possible hostiles.

6. The drone delivery system of claim 1, wherein perform an environmental scan during transit comprises scan for a landing zone.

7. The drone delivery system of claim 1, wherein the communication logic is further to instruct a second drone to travel ahead of the drone and perform an environmental scan during transit.

8. The drone delivery system of claim 1, wherein perform an environmental scan during transit comprises comparing current environmental data with stored historical environmental data.

9. The drone delivery system of claim 1, further comprising route determination logic to determine a route for an emergency vehicle to follow to the target device based on the environmental scan.

10. The drone delivery system of claim 1, wherein the request signal is generated based on historical use of the package.

11. The drone delivery system of claim 10, wherein the request signal is generated by a computing device remote from the target device.

12. A drone request system, comprising:
condition detection logic to identify a package request event proximate to the drone request system, wherein the package request event is identified based on one or more sensor signals; and
transmitting logic to transmit a request signal indicative of the package request event to a drone delivery system, wherein the drone delivery system is to instruct the drone to carry a package to the drone request system in response to the request signal.

13. The drone request system of claim 12, wherein the package request event is a damage event.

14. The drone request system of claim 12, further comprising a delivery system to deliver a medical treatment to a user proximate to the drone request system in response to identification of the package request event.

15. The drone request system of claim 14, wherein the delivery system is to deliver the medical treatment to the user in response to a command signal from the drone.

16. A reconstructive surgery support system, comprising:
a memory storing a LIDAR scan of a portion of a patient's body prior to a damage event affecting a contour of the portion of the patient's body; and
a visual indication system to project a visual indicator of a pre-damage contour of the portion of the patient's body, on the portion of the patient's body, based on the LIDAR scan.

17. The reconstructive surgery support system of claim 16, wherein the visual indication system comprises:
feedback logic to change the visual indicator as surgical reconstruction is performed on the portion of the patient's body to indicate a difference between the pre-damage contour and a current contour.

18. The reconstructive surgery support system of claim 17, wherein change the visual indicator comprises change the color of the visual indicator.

19. The reconstructive surgery support system of claim 17, wherein the surgical reconstruction comprises the application of a filler.

20. The reconstructive surgery support system of claim 16, further comprising:
surgical implement indicator logic to provide a visual indicator of a location of a surgical implement under the patient's skin during surgical reconstruction.
